(19) United States
(12) Patent Application Publication
(10) Pub. No.: US 2026/0321347 A1
(43) Pub. Date: Oct. 15, 2026

(54) SYSTEM AND METHOD OF PREDICTING A GENE EXPRESSION PROFILE

(71) Applicant: The Open University of Israel, Raanana (IL)

(72) Inventor: Noam SHENTAL, Ness Ziona (IL)

(73) Assignee: The Open University of Israel, Raanana (IL)

(21) Appl. No.: 19/109,283

(22) PCT Filed: Sep. 13, 2023
(86) PCT No.: PCT/IL2023/050995

§ 371 Date: Mar. 6, 2025

Related U.S. Application Data
(60) Provisional application No. 63/406,120, filed on Sep. 13, 2022.

(51) Int. Cl. G16B 25/10; G06F 30/27

(52) U.S. Cl. CPC G16B 25/10; G06F 30/27

(57) ABSTRACT

A system and method of predicting a gene expression profile (GEP) by at least one processor may include receiving a pre-treatment GEP vector, representing a GEP of a target biological sample prior to treatment, in an input GEP space; receiving a treatment identification data element, representing at least one target treatment; and applying a first, pretrained machine learning (ML) model on: (a) the pre-treatment GEP vector and (b) the treatment identification data element, to predict a post-treatment GEP vector, representing an expected GEP following application of the at least one target treatment on the target biological sample, in an output GEP space.

### CROSS REFERENCE TO RELATED APPLICATIONS

This application claims the benefit of priority under 35 U.S.C. § 119 (e) of U.S. Provisional Patent Application No. 63/406,120, filed Sep. 13, 2022. The contents of the above application is all incorporated by reference as if fully set forth herein in its entirety.

### FIELD OF THE INVENTION

The present invention relates generally to the field of assistive diagnosis and treatment. More specifically, the present invention relates to predicting a gene expression profile following treatment.

### BACKGROUND OF THE INVENTION

Currently available active treatments and medications undergo an extensive, and expensive process of research, development and testing, en route of being approved for treatment of specific, designated ailments such as cancer. Strong need is currently felt for methods that may assist in redesignating approved treatments and/or groups thereof, so as to indicate useful treatment of previously untested medical conditions.

### SUMMARY OF THE INVENTION

Embodiments of the invention may include a generative, Machine Learning (ML) based model that may use a given, pre-treatment Genomic Expression Profile (GEP) of a predetermined biological sample, to predict an effect of a predefined treatment on both a post-treatment GEP level and a phenotypic outcome level.

The following Table 1 may be used as a glossary for some of the terms used herein, for the reader's convenience.

TABLE 1

| | |
|---|---|
| Biological | The terms “Biological sample” and “Biological sample type” may |
| sample, | be used herein interchangeably to refer to any type of biological |
| Biological | matter comprising a cellular structure, including for example a |
| sample type | single biological cell, a cell line, a tissue of an organism, an organ |
| | of an organism, a microorganism, and a macro-organism (e.g., an |
| | animal, a human subject, and the like). It may be appreciated that |
| | discussion of comparison between different biological samples may |
| | relate to different samples of the same type (e.g., cells of the same |
| | cell line) or to biological samples of different types (e.g., cells of |
| | different tissues, cells of different cell lines, etc.), according to |
| | context. |
| Gene | The terms “Gene Expression” and “Gene Expression Profile” |
| Expression, | (GEP) may be used herein interchangeably to indicate products of |
| Gene | transcription (e.g., coding and non-coding RNA molecules) and/or |
| Expression | translation (e.g., proteins) derived from activation of one or more |
| Profile | genes in the biological sample, as known in the art. |
| (GEP) | For example, embodiments of the invention have been |
| | experimentally applied on transcriptome profiles, to predict post- |
| | treatment transcriptome profiles, as elaborated herein. In these |
| | experiments, the transcriptome profiles have been provided by the |
| | currently available L1000 technology. |
| | As known in the art, L1000 is a high-throughput gene expression |
| | assay that measures mRNA transcript abundance of approximately |
| | 1000 predefined “landmark” genes from human cells. |
| GEP vector | The terms “GEP” and “GEP vector” may be used herein |
| | interchangeably according to context, to indicate a data structure |
| | (e.g., a vector of elements) representing GEP. For example, a GEP |
| | vector may include a plurality of elements or entries, each |
| | representing an abundance of a product of gene expression. |
| | Pertaining to the example of the L1000 transcriptome, a GEP or |
| | GEP vector may include 1000 elements, where each element |
| | represents abundance of a specific mRNA molecule in the |
| | biological sample. |
| GEP space | The term “GEP space” may be used herein to indicate a |
| | multidimensional space (e.g., multidimensional Euclidean space), |
| | where each axis represents a specific component of a GEP vector. |
| | Pertaining to the same example of the L1000 transcriptome, the |
| | GEP space may be a 1000-dimensional space, defined by 1000 |
| | orthogonal axes, that may accommodate representation of the 1000- |
| | element GEP vectors. |
| Latent vector, | The terms “Latent vector” and “Latent GEP vector” may be used |
| Latent GEP | herein interchangeably to indicate an embedding as known in the |
| vector, | art, of a GEP vector into a representation having reduced |
| Latent space, | dimensionality in relation to that GEP vector. |
| Latent GEP | The terms “Latent space” and “Latent GEP space” may be used |
| space. | herein to indicate a multidimensional space (e.g., multidimensional |
| | Euclidean space), where each axis represents a specific component |
| | of a Latent GEP vector. |
| Treatment, | The term “treatment” or “target treatment” may be used herein to |
| Target | indicate any type of action (or inaction) or combinations thereof, |
| treatment, | that may be applied to a biological sample of interest. |
| Active | A treatment of a biological sample may be referred to as an “active” |
| treatment, | treatment, by applying a target drug to the biological sample, |
| Delay | applying a target dosage of a drug to the biological sample, applying |
| treatment | any type of energy or radiation (e.g., electromagnetic radiation, |
| | acoustic or ultrasound radiation, heat, and the like) to the biological |
| | sample, and/or applying a target chemical or dietary supplement to |
| | the biological sample. |
| | A treatment of a biological sample may be referred to as a “delay” |
| | treatment when no active treatment is applied other than a |
| | predefined delay. In other words, a “delay treatment” may include |
| | allowing a predefined period of time to elapse, without applying any |
| | of the actions mentioned above on the biological sample. |
| Pre-treatment | The terms “Pre-treatment GEP” or “Pre-treatment GEP vector” may |
| GEP, | be used herein interchangeably to indicate a data structure that |
| Post-treatment | represents a GEP (e.g., abundance of products of gene expression) |
| GEP, | pertaining to a specific biological sample, prior to application of |
| Active | treatment (e.g., ovals of FIG. 4B). |
| treatment | The terms “Post-treatment GEP” or “Post-treatment GEP vector” |
| GEP, | may be used herein interchangeably to indicate a data structure that |
| Delay GEP. | represents a GEP (e.g., abundance of products of gene expression) |
| | pertaining to a specific biological sample, after application of |
| | treatment (e.g., circles and squares of FIG. 4B). |
| | The terms “Delay GEP” or “Delay GEP vector” may be used herein |
| | as a type of post-treatment GEP, by which the applied treatment |
| | only includes a predefined delay (e.g., circles of FIG. 4B). |
| | The terms “Active treatment GEP” or “Active treatment GEP |
| | vector” may be used herein as a type of post-treatment GEP, by |
| | which the applied treatment includes active treatment (e.g., |
| | applying a specific drug), with a predefined delay (e.g., squares of |
| | FIG. 4B). |
| Transition, | The terms “Transition”, “GEP transition”, and “GEP Transition |
| GEP Transition, | vector” may be used herein interchangeably to indicate a change in |
| GEP Transition | GEP, following treatment of a biological sample. As elaborated |
| vector. | herein, this transition may be represented as a vector that may |
| | connect between “pre-treatment” GEP (e.g., a pre-treatment GEP |
| | vector of the biological sample) and a “post-treatment” GEP (e.g., |
| | a post-treatment GEP vector of the biological sample). |
| Neural Network | A Neural Network (NN) or an artificial neural network (ANN), e.g., |
| (NN) | a neural network implementing a machine learning (ML) or |
| | artificial intelligence (AI) function, may refer to an information |
| | processing paradigm that may include nodes, referred to as neurons, |
| | organized into layers, with links between the neurons. The links |
| | may transfer signals between neurons and may be associated with |
| | weights. A NN may be configured or trained for a specific task, e.g., |
| | pattern recognition or classification. |
| | Training a NN for the specific task may involve adjusting these |
| | weights based on examples. Each neuron of an intermediate or last |
| | layer may receive an input signal, e.g., a weighted sum of output |
| | signals from other neurons, and may process the input signal using |
| | a linear or nonlinear function (e.g., an activation function). The |
| | results of the input and intermediate layers may be transferred to |
| | other neurons and the results of the output layer may be provided as |
| | the output of the NN. Typically, the neurons and links within a NN |
| | are represented by mathematical constructs, such as activation |
| | functions and matrices of data elements and weights. A processor, |
| | e.g., CPUs or graphics processing units (GPUs), or a dedicated |
| | hardware device may perform the relevant calculations. |
| Autoencoder, | The term “Autoencoder” may be used herein to refer to a type |
| Variational | of ANN that may include a first portion, referred to as an |
| Autoencoders | “Encoder”, and a second portion, referred to as a “Decoder”. The |
| (VAEs) | encoder may be used to learn efficient coding of incoming data, |
| | thereby producing a representation or encoding of that data in a |
| | latent space of reduced dimensionality. The decoder may be adapted |
| | to regenerate the incoming data from the encoded representation, |
| | thereby validating the encoding of the Encoder portion. |
| | A Variational Autoencoder (VAE) is a type of autoencoder, as |
| | known in the art. |

Embodiments of the invention may include a method of predicting a gene expression profile (GEP) by at least one processor. According to some embodiments, the at least one processor may receive a pre-treatment GEP vector, representing a GEP of a target biological sample prior to treatment, in an input GEP space; receive a treatment identification data element, representing at least one target treatment; and applying a first, pretrained ML model on: (a) the pre-treatment GEP vector and (b) the treatment identification data element, to predict a post-treatment GEP vector. As elaborated herein, the post-treatment GEP vector may represent an expected GEP following application of the at least one target treatment (e.g., an active treatment or delay treatment) on the target biological sample, in an output GEP space.

According to some embodiments, the target treatment may include, for example applying a target drug to the biological sample, applying a target dosage of a drug to the biological sample, applying radiation treatment to the biological sample, applying a target dietary supplement to the biological sample, and allowing a predefined period of time to elapse on the biological sample.

The first ML model may include an encoder model, trained to receive at least one GEP vector, represented in the input GEP space, and characterized by a first dimensionality, and transform the input GEP space representation of the at least one GEP vector into a latent space representation, characterized by a second, reduced dimensionality.

According to some embodiments, the at least one processor may be configured to train the ML model by receiving a plurality of pre-treatment GEP vectors, annotated according to corresponding biological sample types; receiving a plurality of post-treatment GEP vectors, annotated according to corresponding biological sample types and applied treatments; and training the encoder model to produce the latent space representation of the received GEP vectors, while constraining the GEP vectors to be clustered, in the latent space representation, to form linearly separable clusters.

According to some embodiments, each cluster may include a representation of one or more GEP vectors, characterized by (a) a common biological sample type, and/or (b) a common applied treatment.

The at least one processor may be configured, for one or more applied treatments, to calculate a plurality of treatment vectors in the latent space, each representing transition between a pre-treatment GEP vector cluster of a specific biological sample type and a post-treatment GEP vector cluster of the same biological sample type, following application of the relevant treatment. The at least one processor may subsequently train the encoder model to encode the GEP vectors into the latent space representation, while further constraining the latent space representation of GEP vectors. Such constraint may include, for example limiting all treatment vectors that represent an effect of a specific treatment (e.g., a specific active treatment, with or without delay), on a variety of different biological samples to be co-linear. Additionally, or alternatively, such constraint may include, for example limiting all application vectors, representing effect of a specific active treatment on a variety of different biological samples to be co-linear in the latent space.

Additionally, or alternatively, the at least one processor may be configured to calculate, for two or more applied treatments, a plurality of treatment vectors in the latent space. Each such treatment vector may represent transition between a pre-treatment GEP vector cluster of a specific biological sample type and a post-treatment GEP vector cluster of the same biological sample type, following application of a specific, relevant treatment. The at least one processor may subsequently train the encoder model to encode the GEP vectors into the latent space representation, while further constraining the latent space representation of GEP vectors such that each treatment of the two or more applied treatments corresponds to a distinct direction of treatment vectors. In other words, the encoder model may be trained such that the effect of different active treatments on any type of biological sample would be represented in the latent space by transition vectors that have maximal a difference in their directionality (e.g., a maximal cosine difference).

Additionally, or alternatively, the at least one processor may receive a plurality of pairs of GEP vectors, where each pair corresponds to a specific biological sample type. Each such pair may include (a) a pre-treatment GEP vector, representing GEP of the relevant biological sample type prior to treatment, and (b) a post-treatment GEP vector, representing GEP of the relevant biological sample type following a specific applied treatment. According to some embodiments, the at least one processor may iteratively train the encoder model to produce the latent space representation of the GEP vectors. Each such iteration may include calculating a treatment vector, representing transition between the pre-treatment GEP vector and a post-treatment GEP vector of a first pair in the latent space; calculating a treatment vector, representing transition between the pre-treatment GEP vector and a post-treatment GEP vector of a second pair in the latent space; and training the encoder model to produce the latent space representation of the received GEP vector such that the treatment vector of the first pair may be aligned with the treatment vector of the second pair.

Additionally, or alternatively, the at least one processor may predict a post-treatment GEP vector of the target biological sample following application of the target treatment by obtaining, from the encoder model, a target treatment vector corresponding to the applied target treatment; associating the treatment vector to the treatment identification data element of the target drug; and applying the encoder model on: (a) the pre-treatment GEP vector of the target biological sample and (b) the treatment identification data element of the target treatment, to obtain a post-treatment GEP vector, representing GEP of the target biological sample following application of the target treatment, in the latent space.

According to some embodiments, the first ML model further may include a decoder model, trained to transform at least one GEP vector from a representation in the latent space, to a representation in the output GEP space. As elaborated herein, the at least one processor may apply the decoder model on the post-treatment GEP vector, to predict the post-treatment GEP vector of the target biological sample, represented in the GEP output space.

According to some embodiments, the at least one processor may predict a post-treatment GEP vector of a target biological sample following application of a set or group of target treatments by: obtaining, from the encoder model, a set of target treatment vectors corresponding to the set of target treatments; computing a sum vector, representing a combination of the treatment vectors of the set of target treatment vectors, in the latent space; associating the sum vector to a treatment identification data element representing the set of target treatments; and applying the encoder model on: (a) the pre-treatment GEP vector of the target biological sample and (b) the treatment identification data element of the set of target treatments, to obtain a post-treatment GEP vector, representing GEP of the target biological sample following application of the set of target treatments, in the latent space.

The at least one processor may subsequently apply the decoder model on the post-treatment GEP vector, to predict a post-treatment GEP vector representation of the target biological sample, following application of the set of target treatments, in the GEP output space.

According to some embodiments, the at least one processor may obtain, from the encoder model, two treatment vectors, corresponding to two respective treatments. The at least one processor may then calculate a similarity metric value (e.g., a cosine similarity metric value), representing a level of similarity of direction between the two treatment vectors, in the latent space, based on difference in directionality of the two treatment vectors. The at least one processor may further produce a notification of treatment similarity, representing similarity in post-treatment GEP between the two treatments, according to the similarity metric value.

According to some embodiments, the at least one processor may be configured to design a treatment combination based on a predefined desired GEP. For example, the at least one processor may receive (a) a pre-treatment GEP vector, representing GEP of a biological sample prior to treatment, and (b) a desired GEP vector, representing a desired GEP of the biological sample following application of treatment. The at least one processor may apply the encoder module to encode the pre-treatment GEP vector and the desired GEP vector into the latent space representation. The at least one processor may then calculate a desired treatment vector, representing transition between the pre-treatment GEP vector and the desired GEP vector in the latent space; and select one or more recommended treatments, according to the recommended treatments' respective treatment vectors and the desired treatment vector.

Additionally, or alternatively, the at least one processor may apply a second, pretrained ML model on the predicted post-treatment GEP vector, to further predict a phenotype of the target biological sample following application of the target treatment.

Embodiments of the invention may include a system for predicting a gene expression profile (GEP). Embodiments of the system may include a non-transitory memory device, wherein modules of instruction code may be stored, and at least one processor associated with the memory device, and configured to execute the modules of instruction code.

Upon execution of the modules of instruction code, the at least one processor may be configured to: receive a pre-treatment GEP vector, representing a GEP of a target biological sample prior to treatment, in an input GEP space; receive a treatment identification data element, representing at least one target treatment; and apply a first, pretrained ML model on: (a) the pre-treatment GEP vector and (b) the treatment identification data element, to predict a post-treatment GEP vector, wherein the post-treatment GEP vector represents an expected GEP, following application of the at least one target treatment on the target biological sample, in an output GEP space.

As known in the art, Connectivity map (CMAP) is a library containing over 1.5M gene expression profiles from ~5,000 small-molecule compounds, and ~3,000 genetic reagents, tested in multiple cell types.

Embodiments of the invention have been trained and tested using the currently available CMAP data as a training set. As shown herein, embodiments of the invention may provide accurate prediction of post-treatment profiles of each certain cell line given post-treatment data of other cell lines. According to some embodiments, training of the ML model is simultaneously performed over a plurality (e.g., ten) different drugs, and may allow predictions of the activity of drug combinations.

Analyzing the systems allows direct biological interpretation of different impacts of the drug, identification of side effects, and effective description of the drug's pharmacodynamics.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for predicting a gene expression profile (GEP) following treatment, according to some embodiments;

FIG. 2 is a block diagram, depicting a system for predicting GEP following treatment, according to some embodiments;

FIG. 3 is a table representing GEP information, such as mRNA levels of genes in specific biological samples (e.g., cell lines), according to some embodiments of the invention;

FIG. 4A is a schematic diagram representing application of at least one treatment on at least one biological sample, in an input GEP space, a latent space and an output GEP space, according to some embodiments;

FIG. 4B is a schematic diagram representing transitions between pre-treatment GEP vector(s) and post-treatment GEP vector(s) in a latent space, according to some embodiments;

FIG. 4C is a schematic diagram representing different types of constraints that may be applied to GEP vectors in the latent space, according to some embodiments;

FIG. 4D is a schematic diagram depicting a process of predicting effect of a treatment on a biological sample according to some embodiments of the invention;

FIG. 4E is a schematic diagram depicting a process of predicting effect of a combination of active treatments on a biological sample, according to some embodiments of the invention;

FIG. 5 is a flow diagram, depicting a method of predicting GEP following treatment, by at least one processor, according to some embodiments of the invention;

FIGS. 6A and 6B represent accuracy of prediction of GEP, following application of a group of active treatments (e.g., five different drugs), by embodiments of the invention; and

FIG. 7 demonstrates identification of drug similarity by a similarity module, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

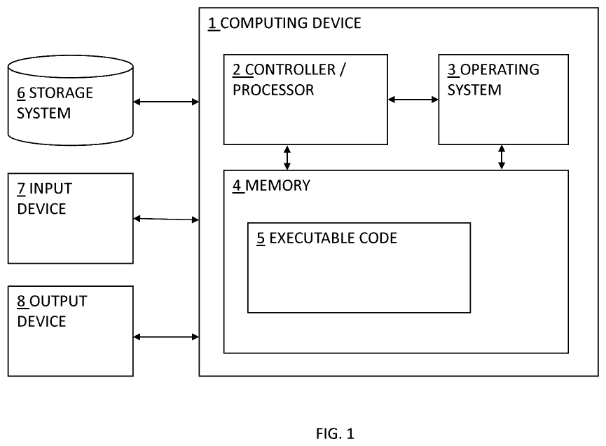

Reference is now made to FIG. 1, which is a block diagram depicting a computing device, which may be included within an embodiment of a system for predicting GEP of a biological sample following a predetermined treatment, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable E code 5 may be any executable code, e.g., an application, a program, a process, task, or script. Executable code 5 may be executed by processor or controller 2 possibly under control of operating system 3. For example, executable code 5 may be an application that may predict GEP of a biological sample following a predetermined treatment, as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to one or more biological samples, and/or one or more predefined treatments may be stored in storage system 6, and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

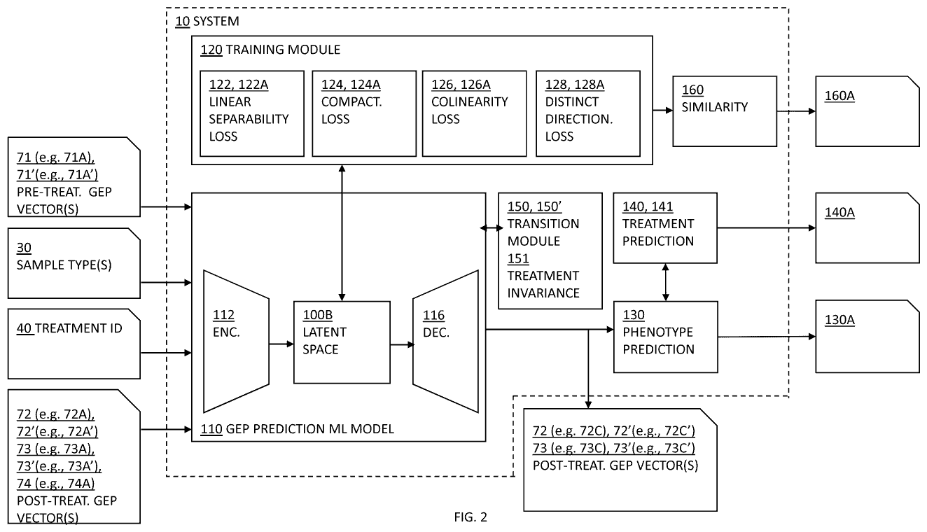

Reference is now made to FIG. 2 which is a block diagram depicting a system 10 for predicting GEP following treatment, according to some embodiments.

According to some embodiments of the invention, system 10 may be implemented as a software module, a hardware module, or any combination thereof. For example, system may be or may include a computing device such as element 1 of FIG. 1, and may be adapted to execute one or more modules of executable code (e.g., element 5 of FIG. 1) to predict GEP of a biological sample following a predetermined treatment, as further described herein.

As shown in FIG. 2, arrows may represent flow of one or more data elements to and from system 10 and/or among modules or elements of system 10. Some arrows have been omitted in FIG. 2 for the purpose of clarity.

As shown in FIG. 2, system 10 may be configured to receive (e.g., via input device 7 of FIG. 1) at least one data element that is a pre-treatment GEP vector 71A, representing GEP of a target biological sample, of a specific sample type 30 prior to treatment, in an input GEP space 100A.

For example, pre-treatment GEP vector 71A (or GEP vector 71A, for short) may pertain to a specific cell line, such as a cell line of a specific cancerous cell (e.g., Melanoma), and may include a plurality of GEP vector elements 71A′. Each GEP vector element 71A′ may represent abundance of a specific gene product. For example, GEP vector 71A may represent a pre-treatment transcriptome of the target biological sample of type 30, such that each GEP vector element 71A′ represents abundance of an mRNA molecule in the pre-treatment target biological sample.

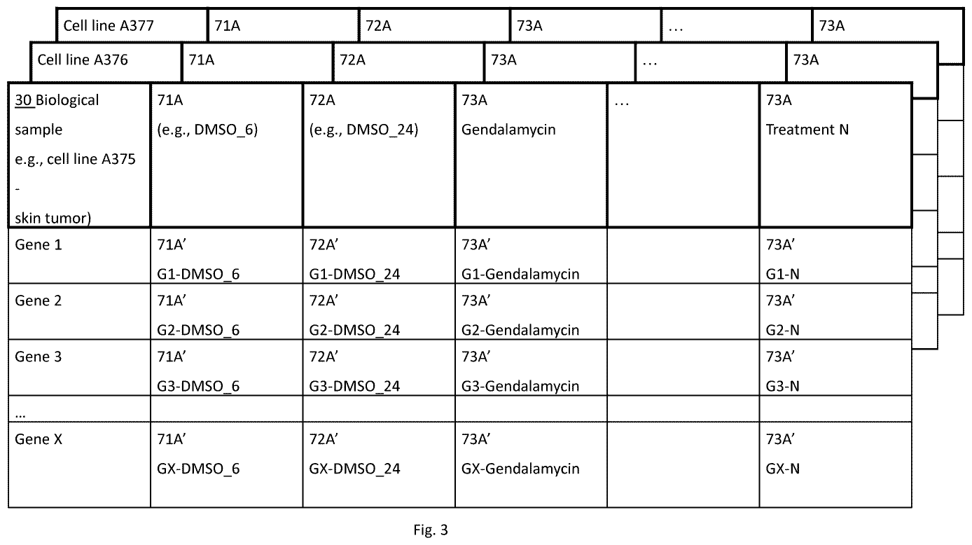

Reference is now made to FIG. 3 which is a table representing GEP information, such as mRNA levels of genes in specific biological samples 30 (e.g., cell lines), according to some embodiments of the invention.

As shown in FIG. 3, GEP information may be formatted in a data structure, such as a table and may be stored in a database (e.g., element 6 of FIG. 1). This GEP information may be divided into portions (e.g., separate tables, as in the depicted example), each pertaining to a specific biological sample 30. FIG. 3 depicts a single such portion, elaborating GEP information pertaining to a specific biological samples 30, e.g., a cell line denoted A375.

In the non-limiting example of FIG. 3, the GEP information of cell line A375 may include a pre-treatment GEP vector 71A (e.g., DMSO-6), which includes a plurality of elements 71A′ ([71A′ G1-DMSO_6, 71A′ G2-DMSO_6, 71A′ G3-DMSO_6, . . . , 71A′ GX-DMSO_6]), each representing GEP (e.g., abundance of mRNA molecules) corresponding to a respective gene of a plurality of genes ([Gene 1, Gene 2, Gene 3, . . . , Gene X]).

Also depicted in the non-limiting example of FIG. 3, the GEP information of cell line A375 may include a pre-treatment (delay) GEP vector 72A (e.g., DMSO-24), which includes a plurality of elements 72A′ ([72A′ G1-DMSO_24, 72A′ G2-DMSO_24, 72A′ G3-DMSO_24, . . . , 72A′ GX-DMSO_24]), each representing GEP (e.g., abundance of mRNA molecules) corresponding to a respective gene of the plurality of genes ([Gene 1, Gene 2, Gene 3, . . . , Gene X]).

Also depicted in the non-limiting example of FIG. 3, the GEP information of cell line A375 may include a post-treatment (active treatment) GEP vector 73A pertaining to treatment of the biological sample by a specific active treatment. In this example, one such active treatment includes administration of Gendalamycin. Each active treatment GEP vector 73A includes a plurality of elements 73A′ ([73A′ G1-Gendalamycin, 73A′ G2-Gendalamycin, 73A′ G3-Gendalamycin, . . . , 73A′ GX-Gendalamycin]), each representing GEP (e.g., abundance of mRNA molecules) corresponding to a respective gene of the plurality of genes ([Gene 1, Gene 2, Gene 3, . . . , Gene X]).

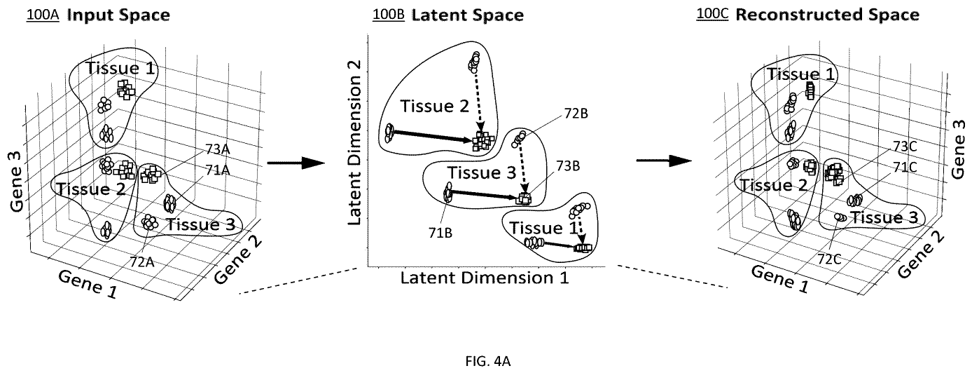

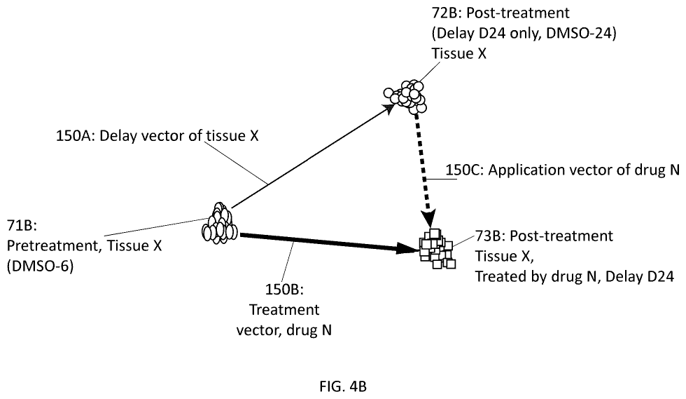

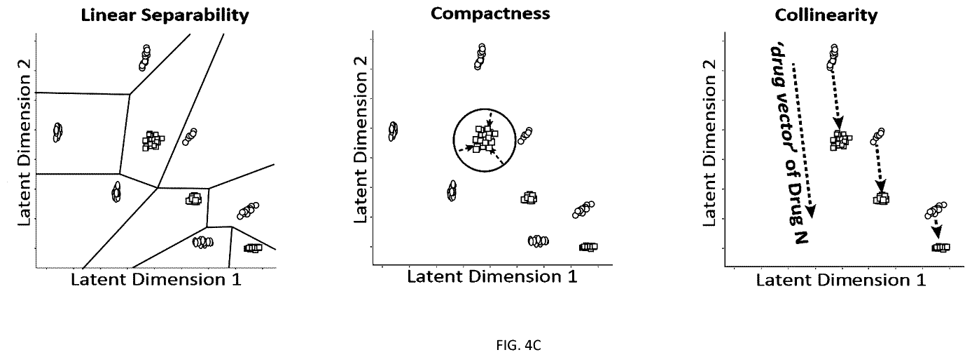

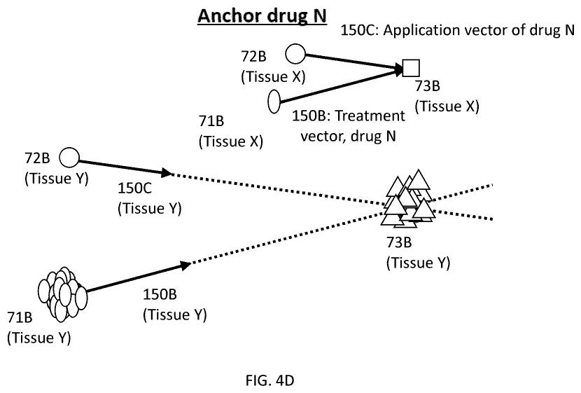

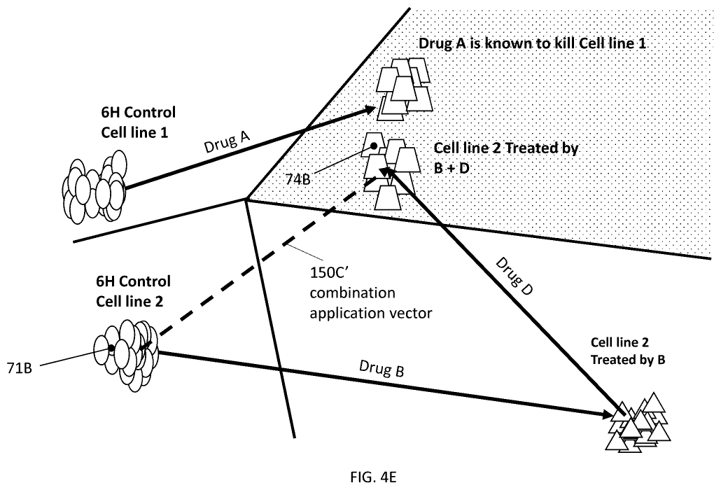

Reference is also made to FIG. 4A which is a simplified, schematic diagram representing application of at least one treatment on at least one biological sample, according to some embodiments.

The left panel of FIG. 4A depicts an example of a simplified, three dimensional, input GEP space 100A, whose axes correspond to abundance of gene products (e.g., transcriptome), of three different genes (denoted “Gene 1”, “Gene 2” and “Gene 3”). Each dot in this example pertains to a single, three dimensional GEP vector, representing a single set of three gene expression abundance measurements. In other words, each of the three elements of each GEP vector (represented by a dot) pertains to measurement of abundance of gene expression of a respective gene (“Gene 1”, “Gene 2” or “Gene 3”).

As also shown in the example of the left panel of FIG. 4A, each group of dots pertains to a specific biological sample type, denoted herein as “Tissue 1”, “Tissue 2” and “Tissue 3”.

According to some embodiments, system 10 may also receive (e.g., via input device 7 of FIG. 1, during an inference stage) a treatment identification data element 40 (also referred to herein as “treatment data 40” or “treatment 40” for short), that may represent at least one target treatment. As elaborated herein, a treatment of interest may include for example application of at least one drug with a predefined dosage, a radiation treatment, a treatment involving a dietary supplement, and any combination of such treatments. In such embodiments, treatment data 40 may, for example, be or include a numeric representation that uniquely identifies the treatment of interest (e.g., a type and dosage of an applied drug).

As depicted in the left panel of FIG. 4A, each biological sample is represented by three different dots (three different GEP vectors), decoded by shape. An oval, representing an instance of pre-treatment GEP vector 71 (e.g., 71A) of the biological sample. A circle represents a post-treatment GEP vector of a delay treatment type, also referred to herein as a delay GEP vector 72 (e.g., 72A), where the treatment (identified by treatment data 40) includes a waiting, without applying active treatment, for a predefined period (e.g., 24 hours). A square represents a post-treatment GEP vector of an active treatment type 73 (e.g., 73A), where the treatment (identified by another treatment data 40) includes application of active treatment (e.g., a specific drug or chemical) to the biological sample, at a predefined dosage, and a predefined delay (e.g., of 24 hours).

This shape coding of ovals, circles and squares is used herein throughout this application to indicate pre-treatment GEP 71 (e.g., 71A, 71B, 71C), post-treatment (delay only) GEP 72 (e.g., 72A, 72B, 72C), and post-treatment (active treatment) GEP 73 (e.g., 73A, 73B, 73C), respectively.

According to some embodiments, system 10 may include a GEP prediction module, which may be, or may include a machine learning (ML) model 110. GEP prediction ML model 110 (or “ML model 110” for short) may be trained to receive input data that includes (a) the pre-treatment GEP vector 71 (e.g., 71A) and (b) the treatment identification data element 40, and predict a post-treatment GEP vector 73 (e.g., 73C), based on the received input data. The predicted post-treatment GEP vector 73 (e.g., 73C) may represent an expected GEP, following application of the at least one target treatment (represented by treatment data 40) on a target biological sample of type 30. As shown on the right pane of FIG. 4A, the predicted post-treatment GEP vector 73C may be included in, or represented by an output GEP space 100C.

For example, ML model 110 may be, or may include a Variational Autoencoder (VAE) Neural Network, that includes an encoder model 112, and a decoder model 116. As elaborated herein, encoder 112 may produce an encoded representation of at least one GEP vector (e.g., pre-treatment GEP vector 71A and/or post-treatment GEP vector 72A/73A) in a latent space 100B of reduced dimensionality.

As shown in the simplified example of FIG. 4A, encoder 112 may be trained to transfer at least one GEP vector (e.g., 71A/72A/73A) from a three-dimensional representation (e.g., “Gene 1”, “Gene 2”, “Gene 3”) of input GEP space 100A to a two-dimensional representation in latent GEP space 100B (denoted 71B/72B/73B, respectively). In other words, encoder model 112 may be trained to receive at least one GEP vector 71A/72A/73A, represented in input GEP space 100A, and characterized by a first dimensionality, and transform the input GEP space representation of the at least one GEP vector 71B/72B/73B into a latent space 100B representation, characterized by a second, reduced dimensionality.

Decoder 116 may be trained to regenerate the incoming data from the encoded representation in latent space 100B, thereby validating the encoding of encoder 112. As shown in the simplified example depicted in the right pane of FIG. 4A, decoder 116 may transfer at least one GEP vector (e.g., 71B/72B/73B) from the two-dimensional representation in latent space 100B to a three-dimensional representation (e.g., “Gene 1”, “Gene 2”, “Gene 3”) in output GEP space 100C (denoted 71C/72C/73C, respectively).

As elaborated herein, encoder 112 may be trained so as to impose specific constraints or restrictions on the geometry of latent space 100B. In some embodiments, these constraints may reflect functional similarity between different drugs. According to some embodiments, system 10 may utilize these constraints to predict an effect of a treatment (e.g., predict post-treatment GEP 72C/73C) following any combination or regimen of treatment.

For example, system 10 may use the constraints on geometry of latent space 100B to predict an effect of a treatment, that includes any unknown or untried combination of drug types, drug concentrations and duration of treatment.

Additionally, or alternatively, system 10 may utilize the constraints on geometry of latent space 100B to extrapolate from one post-treatment GEP 72/73, pertaining to a first set of drugs or treatments, to a post-treatment GEP 72/73 of another set of drugs or treatments.

Reference is now made to FIG. 4B which is a schematic diagram representing transitions between pre-treatment GEP vector(s) 71B and post-treatment GEP vector(s) 72B/73B in latent space 100B, according to some embodiments.

As shown in FIG. 4B, the group of ovals, represent instances of pre-treatment GEP vector 71B of a specific biological sample (“Tissue X”), in the latent space. The group of circles represents instances of post-treatment GEP vector (e.g., a delay GEP vector) 72B of the same biological sample (“Tissue X”) in the latent space, where the treatment (identified by treatment data 40) includes waiting (e.g., not applying active treatment) for a predefined period (e.g., 24 hours). The group of squares represents instances of a post-treatment (e.g., active treatment) GEP vector 73Bin the latent space 100B, where the treatment (identified by another treatment data 40) includes, for example application of a specific drug (denoted “drug N”), at a predefined regimen, and waiting for a predefined period of time (e.g., 24 hours).

According to some embodiments, encoder 112 may receive, for one or more sample types 30 and treatments 40 at least one pre-treatment GEP vector 71A, and produce a latent space 100B representation 71B (e.g., at least one oval) of the at least one pre-treatment GEP vector 71A. Additionally, or alternatively, encoder 112 may receive, for the one or more sample types 30 and treatments 40 at least one post-treatment (e.g., delay treatment) GEP vector 72A, and produce a latent space 100B representation 72B (e.g., at least one circle) of the at least one post-treatment (e.g., delay treatment) GEP vector 72A. Additionally, or alternatively, encoder 112 may receive, for the one or more sample types 30 and treatments 40 at least one post-treatment (e.g., active treatment) GEP vector 73A, and produce a latent space 100B representation 73B (e.g., at least one square) of the at least one post-treatment (e.g., active treatment) GEP vector 73A.

According to some embodiments, system 10 may include a transition calculation module 150 (or “transition 150”, for short), configured to calculate a value of one or more transition vectors (150A, 150B, 150C), representing transitions or changes in GEP vectors (e.g., 71B, 72B, 73B) in latent space 100B.

In some embodiments, transition module 150 may be configured to calculate a transition vector that is a delay vector 150A. Delay vector 150A may represent a first transition of GEP in the latent space: As shown in the example of FIG. 4B, a delay vector 150A in the latent space may be a data element representing transition (e.g., direction and amplitude) from an anchor value, or center of the cluster of ovals (e.g., pre-treatment GEP vectors 71B, or DMSO-6) to an anchor value or center of the cluster of circles (e.g., post-treatment GEP vectors 72B, representing a predefined delay of 24 hours or DMSO-24). In other words, delay vector 150A may represent a mean transition in the GEP of a specific tissue, denoted as tissue X, that is caused by a delay of a predefined period of time (e.g., 24 hours).

Additionally, transition module 150 may calculate a treatment vector 150B, representing transition from a center of the cluster of ovals (e.g., pre-treatment GEP vectors 71B) to a center of the cluster of squares (e.g., post-treatment GEP vectors 73B, representing an effect of active treatment by drug N, and 24 hours delay). In other words, treatment vector 150B may represent a mean transition in the GEP of tissue X, caused by administering drug N and waiting 24 hours.

Additionally, transition module 150 may calculate a third vector, denoted herein as application vector 150C. Application vector 150C may represent transition from a center of the cluster of circles (post-treatment, delay GEP vectors 72B) to a center of the cluster of post-treatment GEP vectors (active treatment GEP) 73B (e.g., treatment by drug N, and waiting 24 hours). In other words, application vector 150C may represent a mean transition in the GEP of tissue X, caused by administering the active treatment (e.g., applying drug N).

Reference is now made to FIG. 4C which is a schematic diagram depicting different types of constraints that may be applied to GEP vectors 71B, 72B, 73B in the latent space, according to some embodiments.

As elaborated herein, system 10 may receive a plurality of GEP vectors in input GEP space 100A such as pre-treatment GEP vectors 71A (ovals), annotated according to corresponding biological sample types 30, and post-treatment GEP vectors 72A (circles) and/or 73A (squares), annotated according to corresponding biological sample types 30 and applied treatments 40.

It may be appreciated that the example of GEP vectors 71A, 72A, 73A in input GEP space 100A depicted in the left pane of FIG. 4A, though illustratively informative, may not convey all possible configurations of GEP vectors 71A, 72A, 73A in that space. For example, input GEP vectors 71A, 72A, 73A may initially not be separable or clustered. In other words input GEP vectors 71A, 72A, 73A may not be confined to multidimensional regions in input GEP space 100A that uniquely correspond to specific biological sample types (e.g., tissues) 30 and/or treatments 40.

According to some embodiments, system 10 may include a training module 120, adapted to train modules of ML model 110 (e.g., encoder 112, decoder 116) based on the plurality of annotated GEP vectors 71A, 72A, 73A, so as to apply at least one constraint on the geometry of latent space 100B. In some embodiments, training module 120 may include one or more loss function calculation modules (122, 124, 126, 128), each adapted to calculate a respective loss function value (122A, 124A, 126A, 128A), according to the annotated GEP vectors 71A, 72A, 73A. Training module 120 may subsequently train encoder 112 (e.g., change one or more weights of encoder 112), so as to minimize a value of the one or more loss function values (122A, 124A, 126A, 128A), thereby imposing the desired constraints on latent space geometry.

For example, training module 120 may include a compactness loss function calculation module 124, configured to calculate a compactness loss function value 124A. The term “compactness” may be used herein to indicate a state in which a group of GEP vectors 71A, 72A, 73A, corresponding to, or annotated by a unique combination of tissue type 30 and treatment type 40 in an N-dimensional latent space 100B, may be confined to an N-dimensional sphere in latent space 100B, as depicted in the example of the middle pane of FIG. 4C.

In other words, compactness loss function value 124A may represent dispersion of samples, according to any appropriate metric (e.g., a Euclidean distance metric) within the multidimensional latent space 100B. Training module 120 of system 10 may train encoder model 112 to minimize a value of compactness loss function value 124A, thereby producing the latent space 100B representation 71B, 72B, 73B of the received GEP vectors 71A, 72A, 73A, while constraining the GEP vectors to be tightly, or compactly clustered.

In another example, training module 120 may include a linear separability loss function calculation module 122, configured to calculate a linear separability loss function value 122A.

The term “linear separability” may be used herein to indicate a state in which each group of GEP vectors 71B/72B/73B, corresponding to, or annotated by a unique combination of tissue type 30 and treatment type 40 in an N-dimensional latent space 100B, may be linearly separable from each other such group of GEP vectors 71B/72B/73B by an (N−1) dimensional plane.

For example, as shown in the left pane of FIG. 4C, each group of dots (e.g., GEP vectors 71B/72B/73B) representing a unique combination of tissue type and treatment (or lack thereof) in a 2-dimensional latent space 100B may be linearly separated from each other group of dots (GEP vectors 71B/72B/73B) by a 1-dimensional plane, i.e., a straight line.

In other words, training module 120 of system 10 may train encoder model 112 to minimize a value of linear separability loss function value 122A, thus producing the latent space 100B representation of the received GEP vectors 71B/72B/73B, while constraining the GEP vectors 71B/72B/73B to be clustered within the latent space representation 100B, to form linearly separable clusters. Each cluster in latent space 100B may include a representation of one or more GEP vectors 71B/72B/73B, characterized by (a) a common biological sample type 30, and/or (b) a common applied treatment 40. For example, linear separability loss module 122 may calculate linear separability loss function value 122A as a number of samples that were incorrectly classified following training of a multiclass perceptron over the latent space, and training module 120 may train encoder model 112 to minimize separability loss function value 122A.

In another example, training module 120 may include a collinearity loss function calculation module 126, configured to calculate a collinearity loss function value 126A.

As depicted in the example of the right pane of FIG. 4C, the term “collinearity” may be used in this context to indicate a state in which a direction of a group of vectors is substantially aligned within multidimensional latent space 100B. In other words, treatment vectors 150B and/or application vectors 150C that pertain to a specific treatment type 40 (e.g., a specific duration of delay, and/or a specific active treatment such as application of a specific drug (denoted “Drug N”) may be referred to as collinear, as their direction may be substantially parallel in respect to the dimensions of latent space 100B.

As elaborated herein (e.g., in relation to FIG. 4B) transition module 150 may calculate a plurality of treatment vectors 150B and/or application vectors 150C, each corresponding to one or more combinations of applied treatments 40 and sample types 30. Each treatment vector 150B may represent transition between a pre-treatment GEP vector 71B cluster (ovals) of a specific biological sample type 30 and a post-treatment GEP vector cluster 73B (squares) of the same biological sample type, following application of the relevant treatment and a predefined delay (e.g., 24 hours). Each application vector 150C may represent transition between a post-treatment, delay GEP vector 72B cluster of a specific biological sample type 30 (circles), following a predefined delay (e.g., 24 hours), and the post-treatment GEP vector cluster 73B (squares) following application of the relevant treatment and the predefined delay.

According to some embodiments, training module 120 of system 10 may train encoder model 112 to encode incoming GEP vectors 71A/72A/73A into the latent space representation (as 71B/72B/73B respectively), while further constraining the latent space representation of GEP vectors 71B/72B/73B such that all treatment vectors 150B that pertain to a specific applied treatment 40 and/or delay, and relate to various biological samples 30, are colinear.

Additionally, or alternatively, encoder model 112 to encode incoming GEP vectors 71A/72A/73A into the latent space representation, while further constraining the latent space representation of GEP vectors 71B/72B/73B such that all application vectors 150C that pertain to a specific applied treatment 40 and/or delay, and relate to different biological samples 30 are colinear.

Taking treatment vectors 150B as an example (also applicable to delay vectors 150A and application vectors 150C): Collinearity loss module 126 may compare treatment vector 150B (and/or vectors 150A/150C) of the same drug over different biological samples. In some embodiments, collinearity loss module 126 may calculate collinearity loss function value 126A as a difference in angle or orientation of these vectors 150B (150A/150C) in the multidimensional latent space 100B. Collinearity loss module 126 may do so separately, for each treatment 40 (e.g., drug), and/or for each combination of a delay and an active treatment (e.g., an applied drug). Training module 120 may collaborate with collinearity loss module 126, to penalize large collinearity loss function values 126A (large differences in orientation) between vectors 150B that pertain to the same treatment and/or delay combinations. Training module 120 may thereby train encoder model 112 to minimize these differences. In other words, training module 120 may train encoder model 112 to minimize a value of collinearity loss function value 126A, to produce the latent space 100B representation 71B/72B/73B of the received GEP vectors 71A/72A/73A, while constraining delay vectors 150A, treatment vectors 150B and/or application vectors 150C that pertain to a specific applied treatment 40 (e.g., invariant of biological sample type 30) to be colinear.

According to some embodiments, training 120 may train encoder 112 to impose the desired constraints on latent space geometry 100B in an iterative process.

For example, system 10 may receive a plurality of pairs of GEP vectors, wherein each pair corresponds to a specific biological sample type 30, and may include (a) a pre-treatment GEP vector 71A, representing GEP of the relevant biological sample 30 type prior to treatment, and (b) a post-treatment GEP vector 72A/73A, representing GEP of the relevant biological sample type following a specific applied treatment. Training module 120 may iteratively train encoder 112 to produce the latent space 100B representation 71B/72B/73B of the received GEP vectors 71A/72A/73A, in an iterative process.

In each iteration of the iterative process, transition module 150 may calculate at least one transition vector, pertaining to a first pair. The at least one transition vector may include a treatment vector 150B representing transition between a pre-treatment GEP vector 71B and a post-treatment GEP vector 72B/73B of a first pair in the latent space, in response to administering a specific treatment (e.g., drug) 40, and/or a predefined delay (e.g., 24 hours). Additionally, or alternatively, the at least one transition vector may include an application vector 150C representing the effect of administering an active treatment (e.g., a drug), as elaborated herein (e.g., regardless of the predefined delay). Transition module 150 may then similarly calculate at least one transition vector (e.g., a treatment vector 150B, an application vector 150C) representing transition between a pre-treatment GEP vector 71B and a post-treatment GEP vector 72B/73B of a second pair in latent space 100B. Training module 120 may subsequently train encoder 112 in each iteration of the iterative process, by changing a value of at least one weight of encoder model 112. In other words, training module 120 may iteratively train encoder 112 to produce a latent space representation 71B/72B/73B of the received GEP vectors 71A/72A/73A such that the transition vectors (e.g., application vector 150C and/or treatment vector 150B) of the first pair are aligned with, or are collinear with corresponding transition vectors (e.g., application vector 150C and/or treatment vector 150B) of the second pair.

It may be appreciated that the constraint of collinearity may facilitate a representation of GEP vectors 20/50 that represents effect of specific treatments (e.g., drugs) 40 in a manner that is invariant to biological sample types 30. In other words, the representation of a direction of transition between pre-treatment GEP vectors 20 and post-treatment GEP vectors 50 (e.g., 50B, 50C) in latent space 100B may be dependent upon the relevant treatment or drug of interest 40, and may be independent of, or invariant to the biological sample or tissue type 30 to which that treatment 40 is applied.

As elaborated herein, such invariance to biological sample type 30 may provide an improvement in assistive diagnosis technology by allowing embodiments of the invention to perform a variety of novel predictions, as elaborated herein.

Reference is also made to FIG. 4D, which is a schematic diagram depicting a process of predicting effect of a treatment on a biological sample according to some embodiments of the invention.

As shown in FIG. 4D, system 10 may exploit the constraint of collinearity to extrapolate a known effect (e.g., a known change or transition in GEP) of a specific treatment 40 on a first biological sample type 30 (e.g., denoted “Tissue X”), as represented by a first application vector 150C or treatment vector 150B, to predict an effect (e.g., a change in GEP) of the same treatment 40 on a second biological sample type 30 (e.g., denoted “Tissue Y”).

In such embodiments, and as shown in the upper pane of FIG. 4D, encoder 112 may encode control samples of pretreatment GEP 71A (e.g., DMSO_6, ovals), and delay treatment GEP 72A (e.g., DMSO_24, circles) into latent space 100B representation, to produce vectors 71B, 72B, respectively. Additionally, encoder 112 may encode anchor points of a drug of interest (73A, squares), to produce vectors 73B respectively, as elaborated herein. Transition module 150 may subsequently calculate treatment vector 150B and application vector 150C based on vectors 71B, 72B, 73B, as elaborated herein.

To predict an unknown effect of the drug of interest on another biological sample (e.g., tissue Y), encoder 112 may encode anchor points GEP 71A (e.g., DMSO_6, ovals), and delay treatment GEP 72A (e.g., DMSO_24, circles) of tissue Y, to create vectors 71B, 72B pertaining to tissue Y. Transition module 150 may then shift application vector 150C and treatment vector 150B to the newly encoded location of the reference points 71B and 72B of the new tissue of interest (tissue ‘Y’). The intersection of these two vectors may define the vectors lengths, to predicting a location of GEP 73B in the latent space, marked herein by triangles. It may be appreciated that this location may predict an effect of an active treatment of interest (e.g., a drug) on a new tissue Y, in the latent space. Decoder 116 may subsequently decode predicted samples, to produce a gene expression profile 73C of the active treatment of interest on the new tissue of interest (tissue ‘Y’).

In another example, system 10 may exploit the constraint of collinearity to predict an effect (e.g., a change in GEP) of a combination or set of active treatments (e.g., a set of drugs) having a plurality of member treatments 40, by combining or adding-up the vectoral representation of application vectors 150C or treatment vectors 150B that pertain to each member treatment 40, in latent space 100B.

For example, transition module 150 may receive a plurality of vectors (e.g., a plurality of application vectors 150C, or a plurality of treatment vectors 150B) each representing transition of GEP of a biological samples 30, following application of a respective active treatments 40 in the latent space 100B, as elaborated herein. Transition module 150 may combine, or sum the plurality of vectors (e.g., by performing Euclidean summation) to produce a combined vector 150′ (e.g., a combined application vector 150C′ or combined treatment vector 150B′), representing transition of GEP, following application of a respective plurality of active treatments 40 in the latent space 100B. Transition module 150 may subsequently shift combined vector 150′ (combined treatment vector 150B′ or combined application vector 150C′) to an anchor GEP vector (e.g., 71B or 72B, respectively) of a new biological sample of interest 30, to predict GEP 73B of the biological sample of interest 30 in latent space 100B. Decoder 116 may subsequently decode GEP 73B, to produce a gene expression profile 73C of the combination of active treatments of interest on the new tissue of interest (tissue ‘Y’).

Additionally, or alternatively, embodiments may predict a post-treatment GEP vector 73C of a target biological sample 30 following application of a set of target treatments 40. In such embodiments, transition module 150 may obtain from encoder model 112 a set of target treatment vectors 150B corresponding to the set of target treatments, and may compute a sum vector 150B′, representing a combination of the treatment vectors 150B of the set of target treatment vectors, in latent space 100B. Transition module 150 may associate the sum vector 150B′ to a treatment identification data element 151 representing the set or combination of target treatments. In a subsequent inference stage, embodiments of the invention may: (a) apply encoder model 112 on pre-treatment GEP vector 71A of the target biological sample to obtain latent space representation 71B, (b) apply the treatment identification data element 151 on the set of target treatments 40 to identify sum vector 150B′ associated with the set of target treatments 40; (c) translate sum vector 150B′ to the latent space representation 71B in latent space 100B, to obtain a post-treatment GEP vector 73B, representing GEP of the target biological sample following application of the set of target treatments, in latent space 100B; and (d) apply a decoder module 116 on post-treatment GEP vector 73B to obtain a post-treatment GEP vector 73C, representing GEP of the target biological sample 30 following application of the set of target treatments 40, in GEP space 100C.

According to some embodiments, system 10 may include a treatment prediction module 140, adapted to design, or produce a recommendation 140A of a treatment combination based on a desired phenotypic output (e.g., a desired GEP). Additionally, or alternatively, treatment prediction module may produce a recommendation of a combination of active treatments 140A, to induce a desired phenotype.

Reference is also made to FIG. 4E, which is a schematic diagram depicting a process of predicting effect of a combination of active treatments on a biological sample, according to some embodiments of the invention.

As shown in the example of FIG. 4E, and as elaborated further below, the latent space 100B tessellation may facilitate marking or mapping of areas representing a predetermined phenotype, as expressed by post-treatment GEP 73C, back into latent space 100B. In the non-limiting example of FIG. 4E, a desired phenotype may include induction of cell death in a target biological sample (e.g., a cell-line of cancerous cells) for different tissues. The “death areas” are schematically represented in FIG. 4E by dotted areas.

It may be appreciated that such mapping of predetermined phenotypes may infer or predict efficiency of certain drugs (e.g., drug A) to produce a desired phenotypic outcome (e.g., kill cell line 1). Additionally, or alternatively, system 10 may utilize mapping of predetermined phenotypes to find a combination of drugs that will ‘move’ a cell line into a region of desired phenotypic outcome (e.g., a “death area”) in latent space 100B.

According to some embodiments, treatment prediction module 140 may receiving from encoder (a) a pre-treatment GEP vector 71A representing GEP of a biological sample prior to treatment, and (b) a desired GEP vector 74A, representing a desired location or transition of GEP of the biological sample, following application of a sought treatment or treatment combination 40.

As elaborated herein, encoder module 112 may encode pre-treatment GEP 71A vector to a pre-treatment GEP vector 71B in the latent space. Additionally, encoder module 112 may encode the desired vector GEP 74A into the latent space representation 74B. Pertaining to the example of FIG. 4E, desired GEP vector 74B may be a GEP vector represented as a location in the “death area” of latent space 100B. The sought treatment or treatment combination 40 should be defined as one that will transfer pre-treatment GEP vector 71B to desired GEP vector 74B, in the latent space 100B.

According to some embodiments, treatment prediction module 140 may calculate a desired treatment vector, representing transition between the pre-treatment GEP vector 71B and the desired GEP vector 74B in the latent space. Treatment prediction module 140 may then collaborate with transition modules to select one or more recommended treatments 40, according to the recommended treatments' respective treatment vectors and the desired treatment vector.

For example, treatment prediction module 140 may select a specific combination of one or more treatment identification data elements 151, associated with respective treatment vectors 150B. This combination treatment identification data elements 151 may be selected so that addition or superposition of respective treatment vectors 150B would transit pre-treatment GEP vector 71B to desired GEP vector 74B in latent space 100B, as depicted by the dashed arrow of FIG. 4E.

In yet another example, training module 120 may include a directionality loss function calculation module 128, configured to calculate a directionality loss function value 128A. The term “directionality” may be used in this context to indicate an extent to which a direction of a specific vector (e.g., 150B, 150C) is distinct from other vectors (e.g., 150B, 150C) in multidimensional latent space 100B. In other words, a directionality value may represent a difference (e.g., a cosine distance) between a direction of a first application vector 150C or treatment vector 150B, pertaining to a first treatment 40, and a direction of a second application vector 150C or treatment vector 150B, pertaining to a second treatment 40, in latent space 100B.

In such embodiments, transition module 150 may calculate, for two or more applied treatments 40, a plurality of treatment vectors 150B and/or application vectors 150C, in latent space 100B. As elaborated herein, each treatment vector 150B may represent transition between a pre-treatment GEP vector 71B cluster (ovals) of a specific biological sample type 30 and a post-treatment GEP vector cluster 73B (squares) of the same biological sample type, following application of the relevant treatment and a predefined delay (e.g., 24 hours). Each application vector 150C may represent transition between a post-treatment GEP vector 72B cluster of a specific biological sample type 30 (circles), following a predefined delay (e.g., 24 hours), and the post-treatment GEP vector cluster 73B (squares) following application of the relevant treatment and the predefined delay. Training module 120 may subsequently train encoder model 112 to encode the GEP vectors 71A/72A/73A into the latent space representation 100B, while further constraining the latent space representation of GEP vectors 71B/72B/73B such that each treatment 40 of the two or more applied treatments will correspond to a distinct direction (e.g., an angle or orientation) of treatment vectors in latent space 100B.

In other words, for each pair of transition vectors 150B/150C, each pertaining to different treatments 40, training module 120 may calculate a directionality value (e.g., a cosine distance), representing similarity of direction between each member vector of that pair. Additionally, or alternatively, training module 120 may calculate a directionality loss function value, based on the directionality value (e.g., cosine distance). Training module 120 may then train encoder 112 (e.g., change one or more weights of encoder 112), to minimize a value of directionality loss function 128A, thereby imposing the desired constraints on latent space geometry 100B, and maximizing distinction of directionality among vectors 150B/150C pertaining to different treatments 40.

It may be appreciated that the constraint of distinct directionality among transition vectors 150B/150C may enhance an association between direction of specific transition vectors 150B/150C and their corresponding treatments 40 in latent space 100B. In other words, the combination of training encoder 112 to minimize both collinearity loss function 126A and directionality loss function 128A as elaborated herein may provide a synergistic effect, that further enhances invariance of GEP vectors 71B/72B/73B and transition vectors 150B/150C to biological sample types 30.

As elaborated herein, ML model 100 may include a decoder model 116, trained to transform at least one GEP vector (e.g., 71B/72B/73B) from a representation in the latent space 100B, to a representation (71C/72C/73C, respectively) in the output GEP space 100C. For example, as depicted in the right pane of FIG. 4A, decoder 116 may be trained (e.g., parallel to training of encoder 112) on one or more input data samples (e.g., one or more GEP vectors 71A/72A/73A) to reproduce the one or more input data samples in the output GEP space 71C/72C/73C. Embodiments of the invention may apply the decoder model 116 on post-treatment GEP vector 73B, to predict the post-treatment GEP vector 73C of the target biological sample 30, represented in the GEP output space 100C.

In other words, decoder 116 may be trained to receive at least one latent-space 100B representation 71B/72B/73B of an input GEP vector 71A/72A/73A, and transform the latent-space 100B representation to an output GEP representation 71C/72C/73C of the at least one input GEP vector 71A/72A/73A. For example, training module 120 may train decoder 116 to produce GEP vectors 71C/72C/73C as a reconstruction of input GEP vector 71A/72A/73A, by calculating a reconstruction error value, representing a difference in reconstruction, and penalizing decoder 116 to as to minimize the reconstruction error value.

According to some embodiments, during an inference stage, ML model may be inferred on, or applied on at least one target biological sample 30 to predict a post-treatment GEP vector 73B/73C of the target biological sample 30, following application of a target treatment 40.

For example, system 10 may receive GEP vectors 71A/72A/73A pertaining to application of a specific target treatment 40 of interest on a first biological sample type 30. System 10 may obtain, from encoder model 112 at least one transition vector (e.g., a target treatment vector 150B or application vector 150C) corresponding to application of the target treatment 40 on the first biological sample type 71A/72A/73A, as elaborated herein.

As explained above, encoder 112 may be trained such that a direction of a transition vector (150B/150C) representing application of a treatment 40 on a biological sample 30 in latent space 100B may represent an effect of the target treatment 40 on any biological sample type 30 (e.g., be invariant to the type of biological sample 30). System 10 may therefore associate the transition vector (e.g., treatment vector 150B, application vector 150C) or at least one parameter (e.g., direction) of the transition vector to the treatment identification data element 40 of the target drug.

For example, system 10 may maintain treatment identification data element 151 as a data structure (e.g., a table, on storage device 6 of FIG. 1) that may associate between specific treatment types 40 (e.g., names of specific drugs) and corresponding parameters (e.g., unit-vector direction) of corresponding transition vectors (e.g., treatment vector 150B, application vector 150C) in latent space 100B. In other words, treatment identification data structure 151 may be included in a database that may maintain information of invariance (e.g., direction in latent space 100B) pertaining to each known treatment type 40.

According to some embodiments, system 10 may apply this information of invariance to new biological samples 30 in the latent space 100B, to predict post-treatment GEP 73C in output GEP space 100C. In other words, system 10 may utilize treatment identification data structure 151 to predict an effect (e.g., transition of GEP) of target treatment 40 on a new, target biological sample 30 of interest: System 10 may (a) apply encoder 112 model on pre-treatment GEP vector 71A of the new target biological sample to obtain latent space representation 71B, (b) utilize treatment identification data structure 151 to identify the treatment vector 150B associated with treatment identification data element 40 of the target treatment, and (c) translate treatment vector 150B to latent space representation 71B to obtain a post-treatment GEP vector 73B as elaborated herein (e.g., in relation to FIG. 4D). Post-treatment GEP vector 73B may thus represent GEP of the target biological sample 30 following application of the target treatment 40, in the latent space 100B.

For example, as depicted in FIG. 4B, system 10 may utilize pre-treatment GEP vector 71B of the target biological sample (oval) and a treatment vector 150B of the target treatment 40 (e.g., from data structure 151) to determine post-treatment GEP vector 73B (square) of the target biological sample 30 following treatment 40.

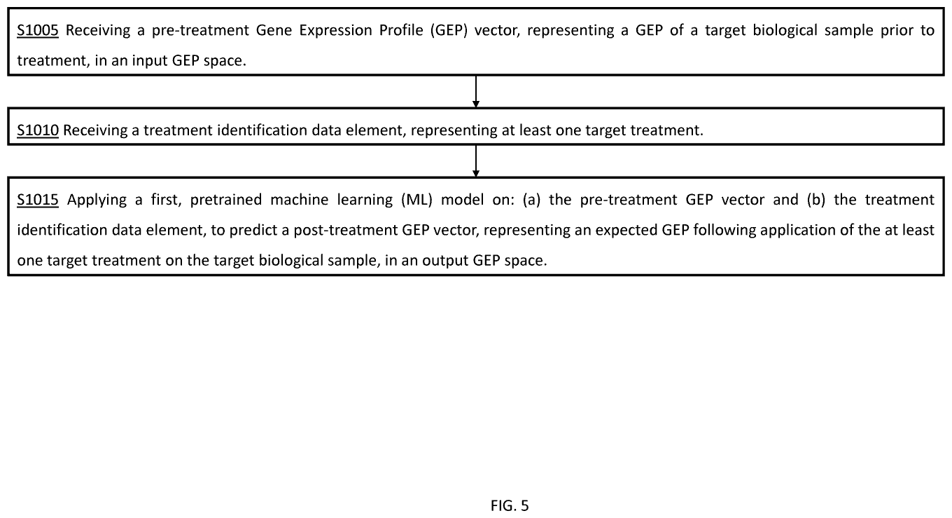

Reference is now made to FIG. 5 which is a flow diagram, depicting a method of predicting GEP following treatment by at least one processor (e.g., processor 2 of FIG. 1), according to some embodiments of the invention.

As shown in step S1005, the at least one processor 2 may receive a pre-treatment GEP vector (e.g., pre-treatment GEP vector 71A of FIG. 2), representing a GEP of a target biological sample (e.g., 30 of FIG. 2) prior to treatment, in an input GEP space 100A.

As shown in step S1010, the at least one processor 2 may receive a treatment identification data element 40, representing at least one target treatment of interest.

As shown in step S1015, the at least one processor 2 may apply a pretrained machine learning model (e.g., GEP prediction model 110 of FIG. 2) on (a) the pre-treatment GEP vector, and (b) the treatment identification data element, to predict a post-treatment GEP vector (e.g., 73C of FIG. 2). As elaborated herein, post-treatment GEP vector 73C may represent an expected GEP, following application of the at least one target treatment 40 on the target biological sample 30, in an output GEP space 100C.

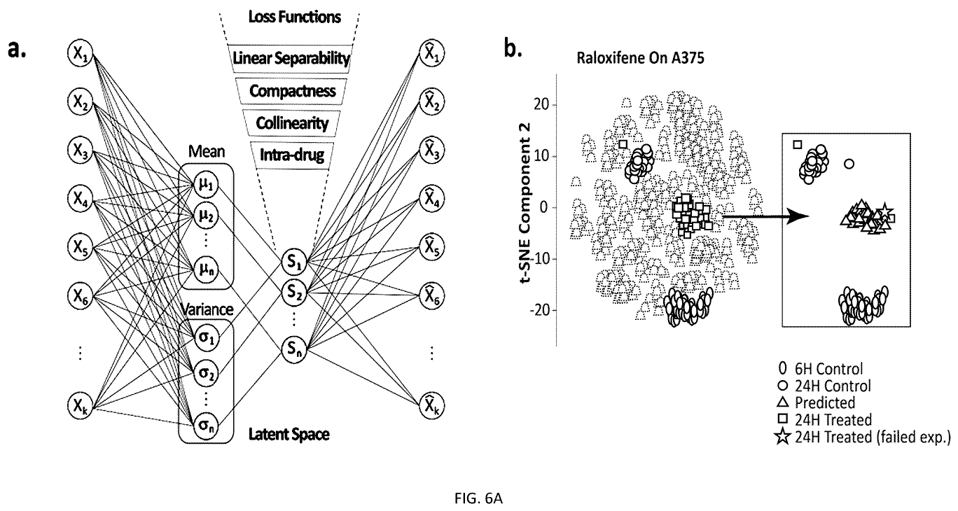

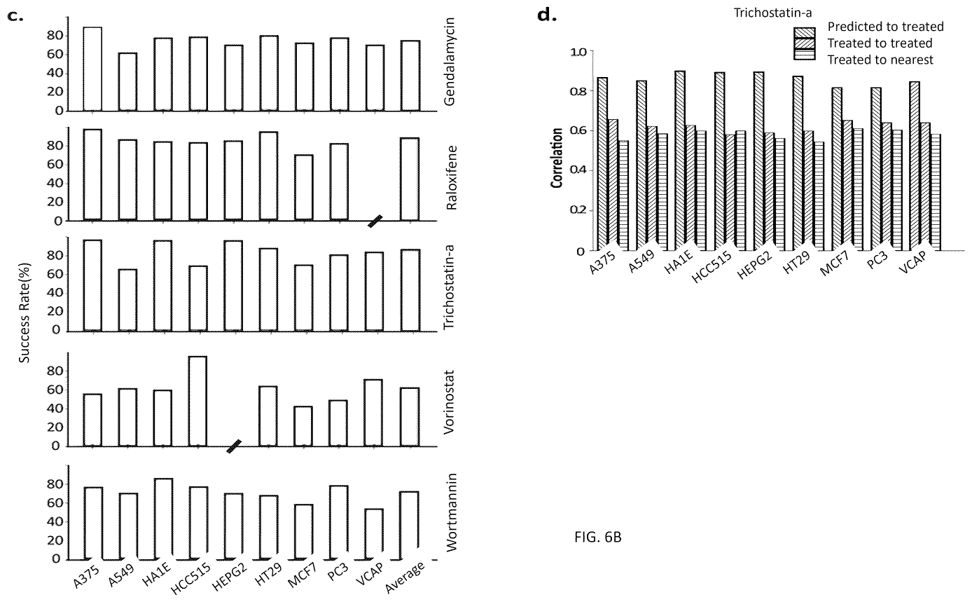

Reference is now made to FIGS. 6A and 6B, which represent accuracy of prediction of GEP, following application of a group of active treatments (e.g., five different drugs), by embodiments of the invention.

As shown on panel A of FIG. 6A, GEP prediction ML model 110 may include a variational autoencoder architecture, that may encode a gene expression vector (e.g., of dimension k=978) into mean (μ) and standard deviation (σ) vectors of dimension n=20.

Panel B of FIG. 6A depicts application of embodiments of the invention on actual GEP data, that was experimentally obtained by applying a set of five active treatments 40 (e.g., drugs) on a group of nine biological samples 30, resulting in 45 combinations. Panel B of FIG. 6A shows a t-distributed stochastic neighbor embedding (t-SNE) visualization of latent space 100B, predicting the effect of a specific treatment of interest 40 (e.g., Raloxifene) over a specific biological sample of interest 30 (e.g., a cell line denoted herein as A375). This visualization produced a plurality of classes, all of which are shown in this image. Maintaining the same shape coding as discussed herein (e.g., as in FIG. 4C), classes of pre-treatment GEP 71B (e.g., DMSO_6) of A375 appear as ovals, and classes of post-treatment, delay GEP 72B (DMSO_24) appear as circles. The treated set of post treatment GEP 73B, where A375 has actually been treated by Raloxifene, appears as squares. Predictions of A375 over Raloxifene (e.g., predicted post-treatment GEP 73C) are depicted as triangles in the right rectangle, together with the same ovals, circles and squares of A375. It may be appreciated that the predicted values (triangles) coincide with the treated set (squares) to a great extent.

Panel C of FIG. 6B depicts success rates in predicting the correct class of treated samples in latent space 100B for each cell line and drug in a leave-one-out fashion. A multi-class linear SVM was trained on the learned latent space of 42 classes and the ‘predicted set’. Data from ‘left-out’ class, i.e., ‘treated set’, were encoded into latent space. Success rate measured the fraction of points that fall in the correct subspace defined the SVM tessellation. Results correspond to an average over ten learning instances for each case. The rightmost column corresponds to the mean success rate over all cell lines for each drug.

Panel D of FIG. 6B depicts Pearson correlation between predicted and treated samples in gene space, for each cell line (bars with forward diagonal lines pattern), shown for Trichostatin-a. Correlations are compared to those calculated between treated samples to themselves (bars with backward diagonal lines pattern), and to the correlation to the closest drug to Trichostatin-a (bars with horizontal lines pattern).

According to some embodiments, system 10 may include a similarity module 160, adapted to predict functional similarity 160A between active treatments (e.g., drugs). Similarity module 160 may be configured to obtain, from encoder model 112, two treatment vectors, corresponding to two respective treatments, and calculate a similarity metric value 160A (e.g., a cosine similarity value), representing a level of similarity of direction between the two treatment vectors, in latent space 100B. Similarity module 160 may subsequently produce a notification of treatment similarity 160A, representing similarity in post-treatment GEP 70B between the two treatments, according to the similarity metric value.

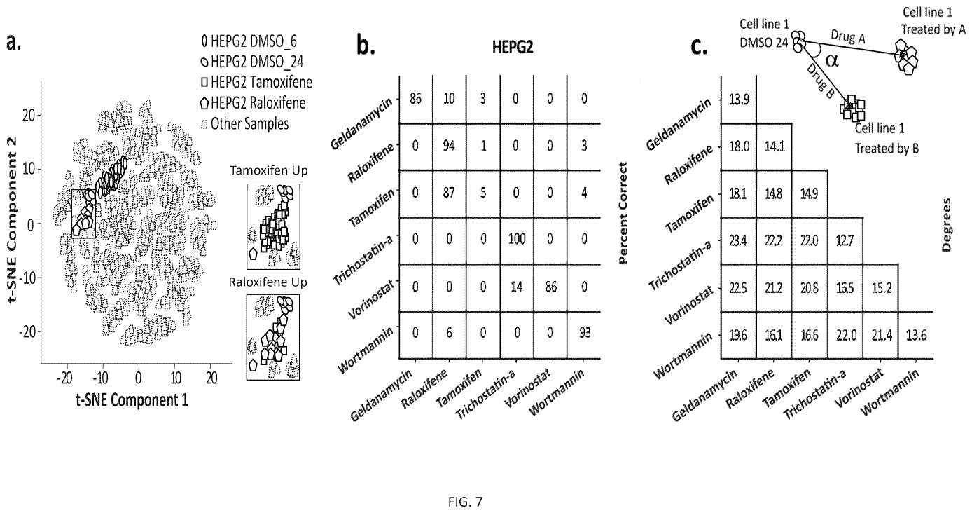

Reference is now made to FIG. 7 which demonstrates identification of drug similarity by similarity module 160, according to some embodiments of the invention.

Panel A of FIG. 7 depicts A t-SNE representation of the latent space tessellation over six active treatments (e.g., drugs) 40, emphasizing classes of a specific biological sample: a cell line denoted HEPG2. These classes include post treatment 73B of HEPG2 by Tamoxifen (squares), post treatment 73B of HEPG2 by raloxifene (pentagons), pre-treatment 71B HEPG2 (e.g., DMSO_6, denoted by ovals), and delay treatment 72B of HEPG2 (24 hour delay, DMSO_24 denoted as circles). It may be observed that HEPG2 Raloxifene and HEPG2 Tamoxifen overlay each other, as shown by the two boxes on the right.

In the embodiment of FIG. 7, a multiclass support-vector network (SVM) was trained on latent space 100B over all cell lines and a set of six drugs. These drugs included five basic-set drugs (Geldanamycin, Raloxifene, Trichostatin-a, Vorinostat and Wortmannin) and Tamoxifen. Panel B of FIG. 7 depicts a confusion matrix, where each row shows the classification rate of HEPG2 treated by a specific drug, in relation to HEPG2 treated by each of the other drugs. The high diagonal value indicates low confusion rates for all drugs except for Tamoxifen, which may regularly be misclassified as Raloxifene.

On panel C of FIG. 7, average angle values between vectors connecting randomly selected pairs of points from delay treatment 72B (e.g., DMSO_24) of a certain cell line to its post treatment (active treatment) 73B points are presented on the diagonal. These are referred to as intra-drug angles. Off diagonal entries where post treatment (active treatment) 73B points were drawn from different drugs (i.e., inter-drug angles). It may be observed that intra-drug angles are consistently smaller than any inter-drug angles for the 5 basic-set drugs. However, the Tamoxifen-Tamoxifen angle is higher than the Tamoxifen-Raloxifene angle, thus indicating similarity in post treatment GEP between Tamoxifen and Raloxifene. It may be therefore appreciated that the metrics of collinearity (e.g., cosine similarity) 126A and distinct direction 124A, discussed herein (e.g., in relation to FIG. 2) may be used by similarity module 160 to produce an indication of functional similarity (e.g., in terms of gene expression) among different active treatments (e.g., drugs).

According to some embodiments, system 10 may include an ML based phenotype prediction model 130, configured to predict a phenotype 130A (e.g., cell death) of a biological target sample, based on gene expression. This predicted phenotype 130A may, in turn, be used by treatment prediction module 140 to produce a recommendation for treatment 140A. Such recommendation for treatment 140A may include a novel application of a known active treatment (e.g., drug) on a new target biological sample 30. Additionally, or alternatively, recommendation for treatment 140A may include usage of a novel combination of active treatments (e.g., drugs) 140A, for treating biological samples of interest (e.g., model cell lines, or even tissues) 30 against predefined medical conditions (e.g., specific types of cancerous tumors).

According to some embodiments, phenotype prediction ML model 130 (or model 130 for short) may be pretrained based on a training dataset to predict a phenotype 130A of a target biological sample, following application of a target treatment.

The training dataset may include a plurality of annotated post-treatment GEP vectors 73C data samples, pertaining to specific biological samples 30 and treatments 40 (or combinations of treatments 40). The post-treatment GEP vectors 73C data samples may be labeled or annotated (e.g., by an expert, via input 7 of FIG. 1) according to respective phenotypic outcomes, including for example cell death (e.g., as depicted in the example of FIG. 4E), treatment toxicity, abundance of specific proteins in the target biological sample 30, and the like.

According to some embodiments, phenotype prediction ML model 130 may collaborate with GEP prediction model 110 to associate or map specific areas or regions in latent space 100B as pertaining to specific phenotypic outcome, as demonstrated herein in relation to the example of cell death, depicted in FIG. 4E.

As elaborated herein, treatment prediction module 140 may subsequently utilize the mapping of predicted post-treatment GEP vectors 73C to corresponding areas or regions in latent space 100B (e.g., as depicted in FIG. 4E) to produce at least one recommendation 140A for a combination 140A of one or more active treatments 40, so as to arrive at a desired phenotypic outcome of the active treatments 40 on a target biological sample 30.

As elaborated herein, embodiments of the invention provide a practical application in the technological fields of assistive diagnostics and pharmaceutics. The benefits provided by the novel representation of treatments as vectors in a latent space as discussed herein may include prediction of previously unknown effects of treatments on specific biological sample types, in both gene expression and phenotypic levels.

For example, embodiments of the invention may provide a prediction of a GEP level, or phenotypic level outcome, in response to application of a specific treatment on a first cell type, based on previous knowledge of effect of that treatment on another cell type.

In another example, embodiments of the invention may provide a prediction of a GEP level, or phenotypic level outcome, in response to application of a specific combination or group of treatments on a first cell type, based on previous knowledge of effect of each treatment of that group, on other cell types. Additionally, or alternatively, embodiments of the invention may provide a recommendation for treatment, composed of one or more active treatments (e.g., drugs), based on the predicted GEP level, or phenotypic level outcome.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of predicting a gene expression profile (GEP) by at least one processor, the method comprising:
receiving a pre-treatment GEP vector, representing a GEP of a target biological sample prior to treatment, in an input GEP space;
receiving a treatment identification data element, representing at least one target treatment;
applying a first, pretrained machine learning (ML) model on: (a) the pre-treatment GEP vector and (b) the treatment identification data element, to predict a post-treatment GEP vector, representing an expected GEP following application of the at least one target treatment on the target biological sample, in an output GEP space.

2. The method of claim 1, wherein the target treatment is selected from a list consisting of: applying a target drug to the biological sample, applying a target dosage of a drug to the biological sample, applying radiation treatment to the biological sample, applying a target dietary supplement to the biological sample, and allowing a predefined period of time to elapse on the biological sample.

3. The method of claim 1, wherein the first ML model comprises an encoder model, trained to receive at least one GEP vector, represented in the input GEP space, and characterized by a first dimensionality, and transform the input GEP space representation of the at least one GEP vector into a latent space representation, characterized by a second, reduced dimensionality.

4. The method of claim 3, wherein training the ML model comprises:
receiving a plurality of pre-treatment GEP vectors, annotated according to corresponding biological sample types;
receiving a plurality of post-treatment GEP vectors, annotated according to corresponding biological sample types and applied treatments; and
training the encoder model to produce the latent space representation of the received GEP vectors, while constraining the GEP vectors to be clustered, in the latent space representation, to form linearly separable clusters.

5. The method of claim 4, wherein each cluster comprises a representation of one or more GEP vectors, characterized by (a) a common biological sample type, and/or (b) a common applied treatment.

6. The method of claim 5, further comprising:
for one or more applied treatments, calculating a plurality of treatment vectors in the latent space, each representing transition between a pre-treatment GEP vector cluster of a specific biological sample type and a post-treatment GEP vector cluster of the same biological sample type, following application of the relevant treatment; and
training the encoder model to encode the GEP vectors into the latent space representation, while further constraining the latent space representation of GEP vectors such that all treatment vectors pertaining to a specific applied treatment are co-linear.

7. The method of claim 5, further comprising:
for two or more applied treatments, calculating a plurality of treatment vectors in the latent space, each representing transition between a pre-treatment GEP vector cluster of a specific biological sample type and a post-treatment GEP vector cluster of the same biological sample type, following application of the relevant treatment; and
training the encoder model to encode the GEP vectors into the latent space representation, while further constraining the latent space representation of GEP vectors such that each treatment of the two or more applied treatments corresponds to a distinct direction of treatment vectors.

8. The method of claim 3, further comprising:
receiving a plurality of pairs of GEP vectors, wherein each pair corresponds to a specific biological sample type, and comprises (a) a pre-treatment GEP vector, representing GEP of the relevant biological sample type prior to treatment, and (b) a post-treatment GEP vector, representing GEP of the relevant biological sample type following a specific applied treatment; and
iteratively training the encoder model to produce the latent space representation of the GEP vectors, wherein each iteration comprises:
calculating a treatment vector, representing transition between the pre-treatment GEP vector and a post-treatment GEP vector of a first pair in the latent space;
calculating a treatment vector, representing transition between the pre-treatment GEP vector and a post-treatment GEP vector of a second pair in the latent space; and
training the encoder model to produce the latent space representation of the received GEP vector such that the treatment vector of the first pair is aligned with the treatment vector of the second pair.

9. The method of claim 7, wherein predicting a post-treatment GEP vector of the target biological sample following application of the target treatment comprises:
obtaining, from the encoder model, a target treatment vector corresponding to the applied target treatment;
associating the treatment vector to the treatment identification data element of the target drug; and
applying the encoder model on: (a) the pre-treatment GEP vector of the target biological sample and (b) the treatment identification data element of the target treatment, to obtain a post-treatment GEP vector, representing GEP of the target biological sample following application of the target treatment, in the latent space.

10. The method of claim 9, wherein the first ML model further comprises a decoder model, trained to transform at least one GEP vector from a representation in the latent space, to a representation in the output GEP space.

11. The method of claim 10, further comprising applying the decoder model on the post-treatment GEP vector, to predict the post-treatment GEP vector of the target biological sample, represented in the GEP output space.

12. The method of claim 7, further comprising predicting a post-treatment GEP vector of a target biological sample following application of a set of target treatments by:
obtaining, from the encoder model, a set of target treatment vectors corresponding to the set of target treatments;
computing a sum vector, representing a combination of the treatment vectors of the set of target treatment vectors, in the latent space;
associating the sum vector to a treatment identification data element representing the set of target treatments; and
applying the encoder model on: (a) the pre-treatment GEP vector of the target biological sample and (b) the treatment identification data element of the set of target treatments, to obtain a post-treatment GEP vector, representing GEP of the target biological sample following application of the set of target treatments, in the latent space.

13. The method of claim 12, wherein the first ML model further comprises a decoder model, trained to transform at least one GEP vector from a representation in the latent space, to a representation in the output GEP space, and wherein the method further comprises applying the decoder model on the post-treatment GEP vector, to predict a post-treatment GEP vector representation of the target biological sample, following application of the set of target treatments, in the GEP output space.

14. The method of claim 7, further comprising:
obtaining, from the encoder model, two treatment vectors, corresponding to two respective treatments;
calculating a similarity metric value, representing a level of similarity of direction between the two treatment vectors, in the latent space; and
producing a notification of treatment similarity, representing similarity in post-treatment GEP between the two treatments, according to the similarity metric value.

15. The method of claim 7, further comprising:
receiving (a) a pre-treatment GEP vector, representing GEP of a biological sample prior to treatment, and (b) a desired GEP vector, representing a desired GEP of the biological sample following application of treatment;
applying the encoder module to encode the pre-treatment GEP vector and the desired GEP vector into the latent space representation;
calculating a desired treatment vector, representing transition between the pre-treatment GEP vector and the desired GEP vector in the latent space; and
selecting one or more recommended treatments, according to the recommended treatments' respective treatment vectors and the desired treatment vector.

16. The method of claim 1, further comprising applying a second, pretrained ML model on the predicted post-treatment GEP vector, to further predict a phenotype of the target biological sample following application of the target treatment.

17. A system for predicting a gene expression profile (GEP), the system comprising: a non-transitory memory device, wherein modules of instruction code are stored, and at least one processor associated with the memory device, and configured to execute the modules of instruction code, whereupon execution of said modules of instruction code, the at least one processor is configured to:
receive a pre-treatment GEP vector, representing a GEP of a target biological sample prior to treatment, in an input GEP space;
receive a treatment identification data element, representing at least one target treatment; and
apply a first, pretrained ML model on: (a) the pre-treatment GEP vector and (b) the treatment identification data element, to predict a post-treatment GEP vector, wherein the post-treatment GEP vector represents an expected GEP, following application of the at least one target treatment on the target biological sample, in an output GEP space.

18. The system of claim 17, wherein the first ML model comprises an encoder model, trained to receive at least one GEP vector, represented in the input GEP space, and characterized by a first dimensionality, and transform the input GEP space representation of the at least one GEP vector into a latent space representation, characterized by a second, reduced dimensionality.

19. The system of claim 18, wherein training the ML model comprises:
receiving a plurality of pre-treatment GEP vectors, annotated according to corresponding biological sample types;
receiving a plurality of post-treatment GEP vectors, annotated according to corresponding biological sample types and applied treatments; and
training the encoder model to produce the latent space representation of the received GEP vectors, while constraining the GEP vectors to be clustered, in the latent space representation, to form linearly separable clusters.

20. The system of claim 19, wherein each cluster comprises a representation of one or more GEP vectors, characterized by (a) a common biological sample type, and/or (b) a common applied treatment.
